(19) United States
(12) Patent Application Publication
(10) Pub. No.: US 2022/0218114 A1
(43) Pub. Date: Jul. 14, 2022

(54) MATTRESS CARE DEVICE

(71) Applicant: LG ELECTRONICS INC., Seoul (KR)

(72) Inventors: JiHyun YOON, Seoul (KR); Hyun Shin KEE, Seoul (KR); Ki Sang JUNG, Seoul (KR); Taihong KIM, Seoul (KR)

(73) Assignee: LG ELECTRONICS INC., Seoul (KR)

(21) Appl. No.: 17/566,290

(22) Filed: Dec. 30, 2021

(30) Foreign Application Priority Data
Jan. 14, 2021 (KR) 10-2021-0005135

(51) Int. Cl. A47C 19/22; A47C 19/04; A61L 2/10; A47L 5/38

(52) U.S. Cl. CPC A47C 19/22; A47C 19/04; A61L 2202/17; A47L 5/38; A61L 2202/26; A61L 2/10

(57) ABSTRACT

The present disclosure provides a mattress care device that may be stored in a hidden state within a bed frame without being exposed to the outside when not in use, and a lift operation part positioned under the mattress for raising the mattress during a sterilization and cleaning of the mattress surface and for lowering the mattress to an initial position.

### CROSS REFERENCE TO RELATED APPLICATION

This application claims the priority benefit of Korean Patent Application No. 10-2021-0005135, filed on Jan. 14, 2021 in the Republic of Korea, which is hereby incorporated by reference in its entirety for all purposes as if fully set forth herein.

### BACKGROUND OF THE INVENTION

### 1. Field of the Invention

The present disclosure relates to a new type of mattress care device that facilitates automatic mattress sterilization while being provided in a bed.

### 2. Description of the Background Art

In general, a mattress is used as a base for bedclothes, and is used on the floor or as a bed.

In the case of a mattress used as a bed, it is provided on a bed frame, and the bedclothes are additionally laid or covered on top of the mattress for a person to sleep.

In the case of the above-mentioned mattress for a bed, continuous management for cleanliness is required because the mattress is provided in a state that is always installed on the bed frame.

Conventionally, a sterilizer having a UV-C lamp on the bottom is provided, with which automatic sterilization and cleaning of a mattress is possible by installing it to be movable in the front-back direction (longitudinal direction of the bed) on the upper side of the mattress.

In this regard, as disclosed in Korean Utility Model Registration No. 20-0388045, Korean Patent Application Publication No. 10-2009-011163, Korean Patent No. 10-1767055, Korean Patent No. 10-1853624, and Korean Patent Application Publication No. 10-2020-0067464, etc.

However, the conventional sterilizer described above has a problem in that a user feels uncomfortable because it is configured to be exposed to the outside of the bed. That is, since cleaning equipment is present in a sleeping place, even if it does not actually smell, it is possible to perceive a smell (bad smell), which may cause serious discomfort.

Moreover, since the sterilizer is exposed to the outside of the bed, there is an inconvenience that interference occurs when the user gets on or off the bed.

Also, the conventional sterilizer is configured to be operated in a state installed on the outer surface of the bed frame, or to be operated in a state installed through the bed frame, and since the part where the sterilizer is installed or installed through is exposed to the outside, there is a disadvantage that not only the aesthetic sense of design is deteriorated, but there is difficulty in design improvement.

In addition, since the conventional sterilizer has a structure exposed to the outside, there is a problem in that foreign substances may be continuously or accidentally caught in the installation or operation part of the sterilizer, and contamination is generated due to accumulation of dust, etc., which causes discomfort to the user.

### DOCUMENTS OF RELATED ART

(Patent Document 0001) Korean Utility Model Registration No. 20-0388045.
(Patent Document 0002) Korean Patent Application Publication No. 10-2009-011163.
(Patent Document 0003) Korean Patent No. 10-1767055.
(Patent Document 0004) Korean Patent No. 10-1853624.
(Patent Document 0005) Korean Patent Application Publication No. 10-2020-0067464.

### SUMMARY OF THE INVENTION

Accordingly, the present disclosure has been made keeping in mind the above problems occurring in the related art, and the present disclosure is intended to provide a new type of mattress care device that may be stored in a hidden state without being exposed to the outside when not in use while facilitating smooth sterilization and cleaning of the mattress surface.

Another objective of the present disclosure is to provide a new type of mattress care device that allows UV-C irradiation to be performed as close to the surface of the mattress as possible to obtain a stable sterilization effect.

Yet another objective of the present disclosure is to provide a new type of mattress care device that may be used on any mattress regardless of mattress thickness.

In order to achieve the above objective, according to an embodiment of the present disclosure, there is provided a mattress care device including a bed frame that may be configured by having an accommodation part with an open top and a storage part with a closed top.

According to the mattress care device of the present disclosure, may be provided a lift operation part moving a mattress up and down.

According to the mattress care device of the present disclosure, a cleaning module may be configured to be stored in the storage part of the bed frame when not in use.

According to the mattress care device of the present disclosure, the cleaning module may be configured to move back and forth (i.e., along a top surface of a mattress, in multiple directions) positioned in the accommodation part when sterilization and cleaning are performed.

According to the mattress care device of the present disclosure, a cleaner moving part configured to move the cleaning module back and forth may be included.

According to the mattress care device of the present disclosure, a controller configured to control operations of the lift operation part, the cleaning module, and the cleaner moving part may be included.

According to the mattress care device of the present disclosure, the bed frame may be configured as a box body with an open top while having a front panel, opposite side panels, and a rear panel.

According to the mattress care device of the present disclosure, an edge part may be formed by protruding inward around a periphery of the open top of the bed frame.

According to the mattress care device of the present disclosure, the storage part may be formed on at least one side of a head-side portion and a foot-side portion of the accommodation part.

According to the mattress care device of the present disclosure, the storage part may be formed extending from the foot-side portion of the accommodation part.

According to the mattress care device of the present disclosure, the storage part may be configured to have a front and rear length for the cleaning module to be completely accommodated therein.

According to the mattress care device of the present disclosure, the lift operation part may be configured to have a lift frame on which the mattress is placed and a lift part for elevating the lift frame.

According to the mattress care device of the present disclosure, the lift part may be configured to have at least one ball screw while being rotated by driving of a screw rotation motor.

According to the mattress care device of the present disclosure, the lift frame may be installed to be raised and lowered by a forward and reverse rotation of the ball screw.

According to the mattress care device of the present disclosure, the lift operation part may operate such that, when sterilization and cleaning is performed, an upper surface of the mattress is positioned at a same height as a bottom surface of the cleaning module or is positioned lower than the bottom surface of the cleaning module.

According to the mattress care device of the present disclosure, the lift operation part may operate such that, when the cleaning module is not in use, an upper surface of the mattress is positioned higher than an upper surface of the bed frame.

According to the mattress care device of the present disclosure, a left and right length of the cleaning module may be configured to be longer than a left and right width of the mattress, and opposite ends of the cleaning module may be respectively connected to opposite side panels in the bed frame.

According to the mattress care device of the present disclosure, an inlet may be formed on a bottom surface of the cleaning module.

According to the mattress care device of the present disclosure, a fan motor for generating a vacuum suction force and a dust collector for collecting foreign substances sucked through the inlet may be provided in the cleaning module.

According to the mattress care device of the present disclosure, the inlet may be formed in a center of a bottom surface of the cleaning module.

According to the mattress care device of the present disclosure, the dust collector may be located on a center in the cleaning module.

According to the mattress care device of the present disclosure, two fan motors may be provided, one on each side of the dust collector.

According to the mattress care device of the present disclosure, a dust bag may be installed replaceably inside the dust collector. That is, the dust bag may be removably installed in the dust collector and may be removed from the dust collector for replacement or cleaning.

According to the mattress care device of the present disclosure, the upper surface of the dust collector may be configured to be open and closed with an open/close cover.

According to the mattress care device of the present disclosure, the dust collector may be configured to be connected to the inlet by a connection duct.

According to the mattress care device of the present disclosure, at least one wall of the cleaning module may be provided with an outlet through which air is discharged.

According to the mattress care device of the present disclosure, a UV-C irradiation part for radiating ultraviolet-c (UV-C) may be provided on a bottom surface of the cleaning module.

According to the mattress care device of the present disclosure, proximity sensors may be located in the front and rear of the cleaning module for detecting proximity to the front and rear surfaces of the bed frame.

According to the mattress care device of the present disclosure, the controller may be configured to control the operation of the cleaner moving unit based on the detection signal sensed by the proximity sensor.

According to the mattress care device of the present disclosure, the cleaner moving part may be configured to include: rack gears provided along opposite side panels of the bed frame; and pinion gears provided on opposite sides of the cleaning module and installed to be engaged with the rack gears respectively, and rotated by receiving a driving force of a gear driving motor to move along the respective rack gears.

According to the mattress care device of the present disclosure, guide parts for supporting both ends of the cleaning module may be provided on the opposite side panels in the bed frame, respectively, and the cleaning module may be configured to move in the front-rear direction of the bed frame by receiving the support of the respective guide parts.

As described above, the mattress care device of the present disclosure has an effect that the cleaning module can be stored without being exposed to the outside when not in use, since the bed frame is formed such that the accommodation part for accommodating the mattress and the storage part for storing the cleaning module are provided separately.

Also, the mattress care device of the present disclosure has an effect that the cleaning module is exposed only when the mattress is being cleaned as the mattress is configured to be raised and lowered inside the accommodation part by the lift operation part provided.

In addition, the mattress care device of the present disclosure has an effect that external exposure of the cleaning module can be prevented when it is not in use since the upper surface of the storage part is formed to be covered from the external environment, by a shielding plate.

Also, the mattress care device of the present disclosure has an effect that the cleaning module can be selectively moved to the accommodation part and to the storage part, since the storage part is formed to connect and communicate with the accommodation part.

In addition, the mattress care device of the present disclosure has an effect that it is possible to adjust the lift height of the mattress because the lift part includes a ball screw and a screw rotation motor, thus the device can be used on any mattress regardless of the thickness thereof.

Also, the mattress care device of the present disclosure has an effect that the cleaning module can clean the mattress by moving from the top of the mattress in the longitudinal direction (i.e., an extension direction of the bed frame) since the top surface of the mattress is positioned equal to or lower than the bottom surface of the cleaning module when the cleaning module is in use.

In addition, the mattress care device of the present disclosure has an effect that the part where the cleaning module is stored can be prevented from being exposed to the outside since the upper surface of the mattress is configured to be positioned higher than the upper surface of the bed frame when the cleaning module is not in use.

Also, the mattress care device of the present disclosure has an effect that the entire upper surface of the mattress can be cleaned since the left and right lengths of the cleaning module are longer than the mattress (i.e., the cleaning module may overlap at least an entire width of the mattress).

In addition, the mattress care device of the present disclosure has an effect that it is possible to clean the mattress to remove foreign substances on the surface of the mattress since the cleaning module is composed of a fan motor and a suction part having a dust collector.

Also, the mattress care device of the present disclosure has an effect that sterilization cleaning to kill bacteria on the surface of the mattress is possible since the UV-C irradiation part that radiates UV-C is provided on a bottom surface of the cleaning module.

In addition, the mattress care device of the present disclosure has an effect that it is possible to control the speed of a front movement and a rear movement of the cleaning module since the cleaner moving part includes rack gears and pinion gears.

Also, the mattress care device of the present disclosure has an effect that the cleaning module is exposed only when in use, since the mattress is installed in a way that it can be moved up and down inside the accommodation part of the bed frame.

In addition, the mattress care device of the present disclosure has an effect that sufficient vacuum suction power is provided regardless of the length of the cleaning module since the dust collector is located in the center of the cleaning module and fan motors are provided on both sides thereof.

Further scope of applicability of the invention will become apparent from the detailed description given hereinafter. However, it should be understood that the detailed description and specific examples, while indicating embodiments of the invention, are given by way of illustration only, since various changes and modifications within the scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention.

FIG. 1 is a perspective view showing a bed to which a mattress care device according to an embodiment of the present invention is applied.

FIG. 2 is a side cross-sectional view showing a bed to which a mattress care device according to an embodiment of the present invention is applied.

FIG. 3 is a perspective view showing a state when the cleaning of the mattress by the mattress care device according to an embodiment of the present invention is performed.

FIG. 4 is a perspective view, different from that of FIG. 3, a state when the cleaning of the mattress by the mattress care device according to an embodiment of the present invention is performed.

FIG. 5 is an exploded perspective view of a mattress care device according to an embodiment of the present invention.

FIG. 6 is a bottom side perspective view of a mattress care device according to an embodiment of the present invention.

FIG. 7 is an enlarged view of “A” in FIG. 2.

FIG. 8 is a cross-sectional view showing the storage structure of the sterilization module of the mattress care device according to an embodiment of the present invention in an unused state.

FIG. 9 is an enlarged view of “B” in FIG. 8.

FIG. 10 is a perspective view showing the peripheral panel of the bed frame removed for explaining the installation structure for the sterilization module of the mattress care device according to an embodiment of the present invention.

FIG. 11 is a view of the main part for explaining the installation structure for the sterilization module of the mattress care device according to an embodiment of the present invention.

FIG. 12 is an enlarged view of “C” in FIG. 11.

FIG. 13 is an exploded perspective view of a sterilization module of a mattress care device according to an embodiment of the present invention.

FIG. 14 is a plan view showing the internal structure of the sterilization module constituting the mattress care device according to an embodiment of the present invention.

FIG. 15 is a plan view showing the cleaner moving part in the sterilization module constituting the mattress care device according to an embodiment of the present invention.

FIG. 16 is a state diagram showing the internal structure of the sterilization module constituting the mattress care device according to an embodiment of the present invention from the front.

FIG. 17 is a block diagram schematically shown to explain the control structure by the controller of the mattress care device according to an embodiment of the present invention.

FIG. 18 is a state diagram showing the internal structure of the sterilization module of the mattress care device according to the embodiment of the present disclosure from the front.

### DETAILED DESCRIPTION OF THE PREFERRED EMBODIMENTS

The present invention will now be described with reference to the accompanying drawings, wherein the same reference numerals have been used to identify the same or similar elements throughout the several views.

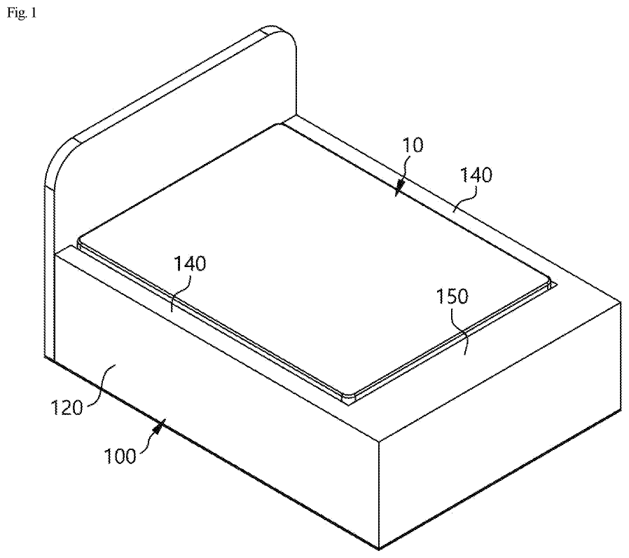

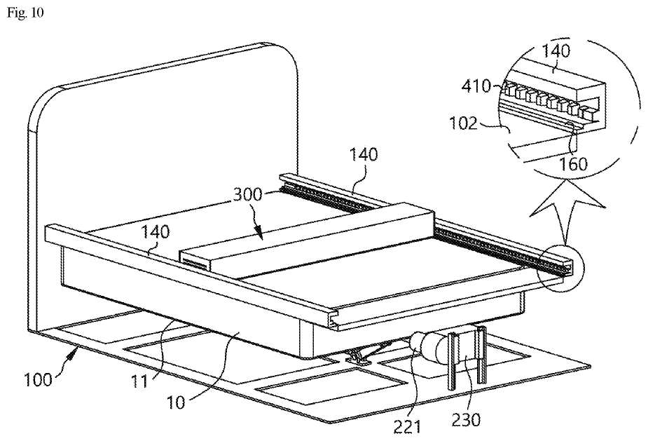

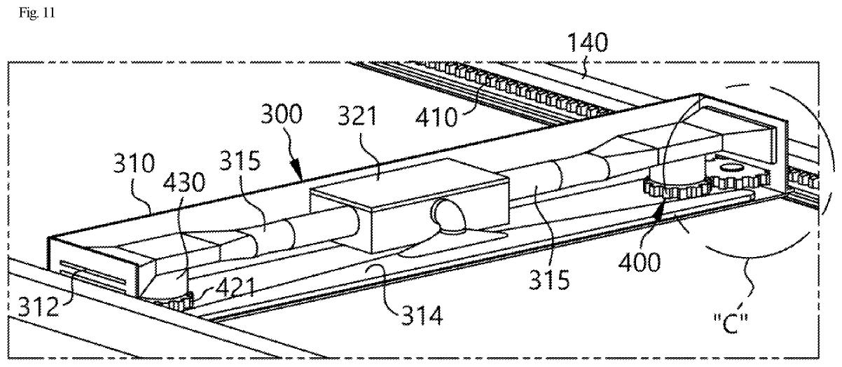

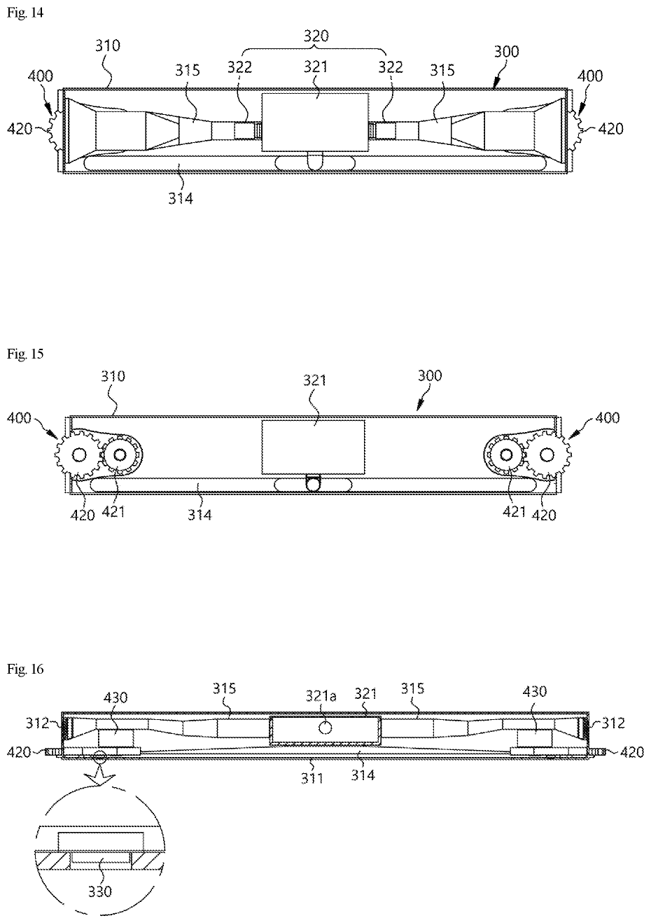

Hereinafter, preferred embodiments of a mattress care device of the present disclosure will be described with reference to the accompanying FIGS. 1 to 18.

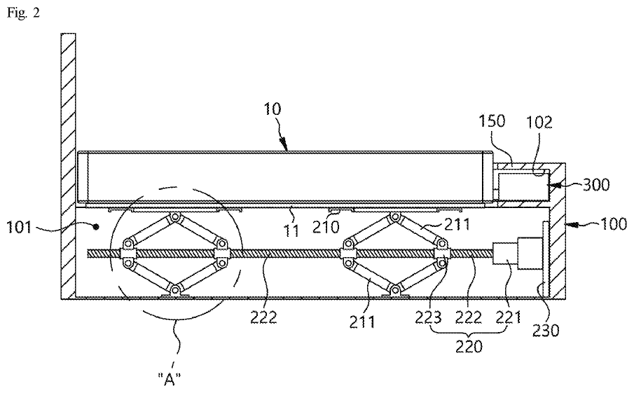

FIG. 1 is a perspective view showing a bed to which a mattress care device according to an embodiment of the present disclosure is applied; and FIG. 2 is a side cross-sectional view showing a bed to which a mattress care device according to an embodiment of the present disclosure is applied.

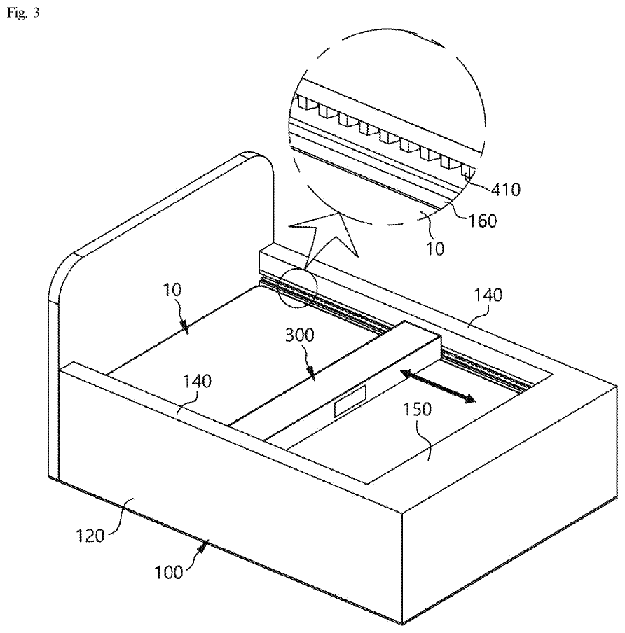

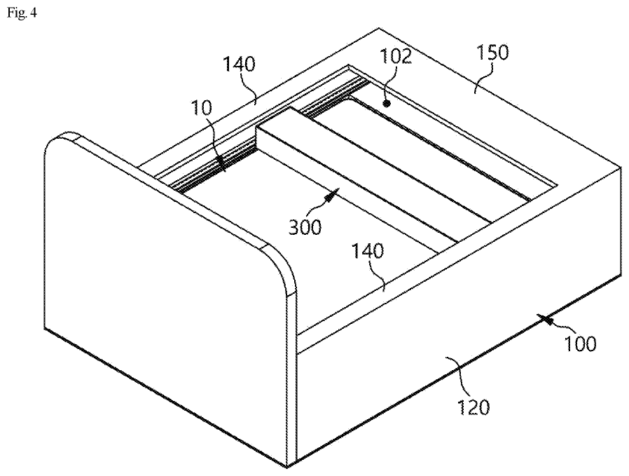

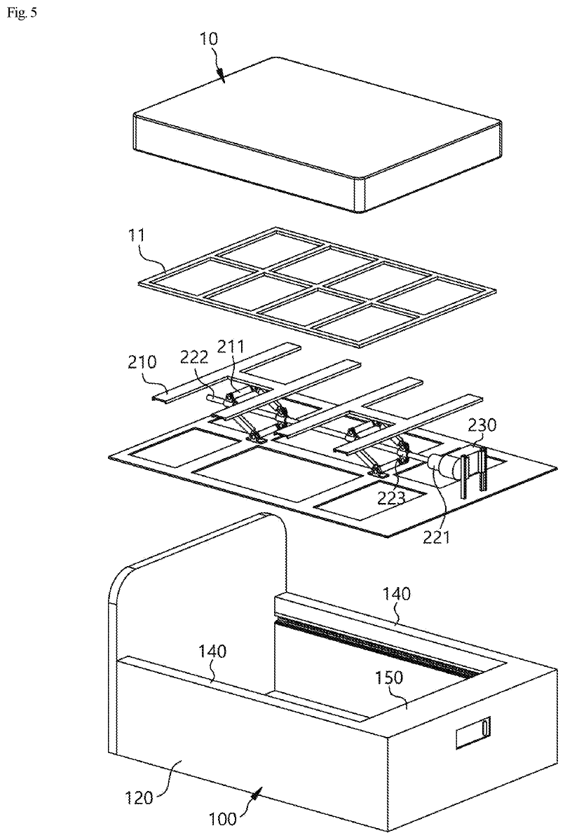

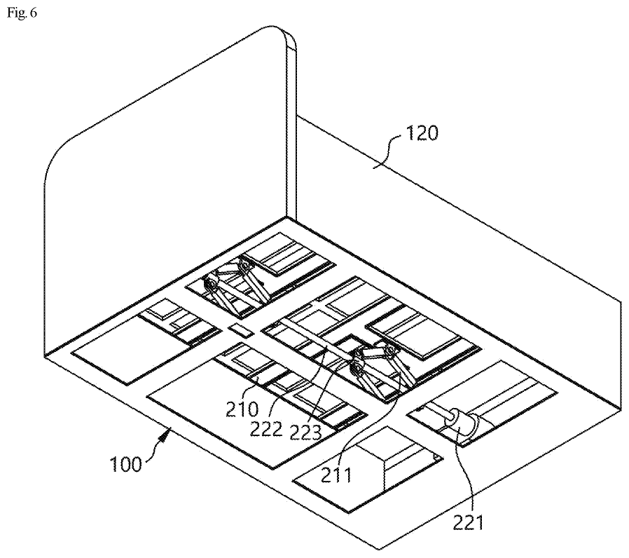

FIGS. 3 and 4 are perspective views showing a state when the cleaning of the mattress by the mattress care device according to an embodiment of the present disclosure is performed; FIG. 5 is an exploded perspective view of a mattress care device according to an embodiment of the present disclosure; and FIG. 6 is a bottom side perspective view of a mattress care device according to an embodiment of the present disclosure.

As shown in these drawings, a mattress care device according to an embodiment of the present disclosure is configured such that, the mattress 10 is installed to be movable down to a position lower than the upper surface of the bed frame 100, and a cleaning module 300 is provided for sterilizing and cleaning while moving on the upper part of the mattress 10 when the mattress 10 is moved downward.

That is, when the sterilization cleaning is not performed, the cleaning module 300 is not exposed to the outside and is kept in a hidden state to prevent a user's visual discomfort.

The mattress care device according to an embodiment of the present disclosure will be described as follows.

First, the mattress care device according to an embodiment of the present disclosure may be configured to include a bed frame 100.

The bed frame 100 is configured to form the exterior of the bed.

The bed frame 100 may be formed as a box body (i.e., a box shape) with an open top while having opposite side panels 120 forming opposite side portions (i.e., a first side panel 120 and a second side panel 120).

Here, an edge part 140 may be formed to protrude inward around the upper surface of the bed frame 100. The edge part 140 serves to cover cleaner moving parts 400 provided at both ends of the cleaning module 300 to be described later so that they are not exposed to the outside. The an edge part 140 may extend from the side panels 120 towards a center of the bed frame 100 and protrude inward around a periphery of the open top of the bed frame.

Of course, the edge part 140 is formed to protrude only a distance that does not interfere with the lifting movement of the mattress 10. That is, the edge part 140 is formed to protrude by a distance in consideration of the left and right width and front and rear length of the mattress 10.

In addition, the bed frame 100 is configured to include an accommodation part 101 and a storage part 102.

The accommodation part 101 is a space from the head-side portion to the foot-side portion of the bed frame 100. That is, the accommodation part 101 extends along a length of the bed frame 100 (i.e., extends in a longitudinal direction of the bed frame 100).

The storage part 102 is a space from the foot-side portion of the accommodation part 101 to the rear panel of the bed frame 100, and the cleaning module 300 may be stored in the storage part 102.

Here, the storage part 102 is provided to be connected and communicated with the rear side of the accommodation part 101, thereby the cleaning module 300 can go back and forth between the storage part 102 and the accommodation part 101.

The storage part 102 includes a shielding plate 150 blocking the internal space while forming an upper surface. That is, the shielding plate 150 prevents the cleaning module 300 stored therein from being exposed to the outside.

In particular, the storage part 102 may be formed to have a front and rear width sufficient to accommodate the cleaning module 300 completely. That is, when the cleaning module 300 is accommodated in the storage part 102, the cleaning module 300 is not exposed to the outside.

Next, the mattress care device according to an embodiment of the present disclosure may be configured to include a lift operation part 200.

The lift operation part 200 is a device provided for elevating the mattress 10.

The lift operation part 200 may be operated such that the upper surface of the mattress 10 is the same height as the bottom surface of the cleaning module 300 or positioned lower than the bottom surface of the cleaning module 300 during sterilization cleaning. Accordingly, even if the position of the cleaning module 300 is not raised, the cleaning module 300 can be moved along the upper surface of the mattress 10.

Also, the lift operation part 200 may operate such that the upper surface of the mattress 10 is positioned higher than the upper surface of the bed frame 100 when the cleaning module 300 is not in use. Accordingly, the cleaning module 300 may be stored in the storage part 102 without being exposed to the outside, thereby preventing the user's visual discomfort.

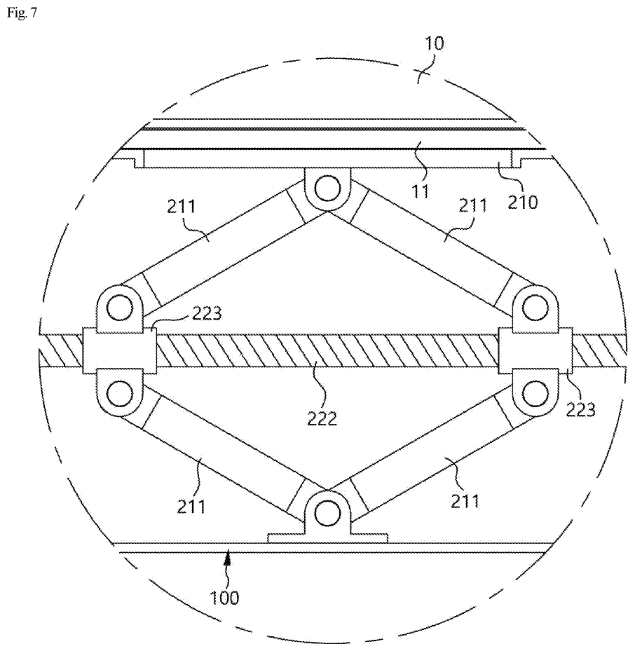

In the embodiment of the present disclosure, the lift operation part 200 is configured to include a lift frame 210 and a lift part 220 as shown in the attached FIGS. 2 and 7.

Here, the lift frame 210 is a portion on which the mattress 10 is placed, and it is preferable that two or more lift frames 210 are provided to support the front side bottom surface and the rear side bottom surface of the mattress 10 at the same time, as illustrated in FIG. 2. Of course, the lift frame 210 may be provided as one (i.e., a unitary member).

The lift part 220 may be configured to include at least one ball screw 222 while being rotated by driving a screw rotation motor 221 as a device for elevating the lift frame 210. That is, the height of the lift frame 210 may be adjusted by the structure using the at least one ball screw 222, thus making the lift frame 210 suitable for use on any mattress regardless of the thickness of the mattress 10.

Here, the lift frame 210 may be installed to be raised and lowered while being connected to at least one link 211 that is folded or unfolded by the forward and reverse rotation of the ball screw 222.

The link 211 may be provided in two or more plural, and each link 211 may be rotatably installed in a plurality of ball housings 223 coupled to a ball screw 222, respectively. That is, a single ball screw 222 may be coupled to a plurality of ball housings 223 to lift the at least one lift frame 210. Here, the ball housings 223 are coupled to the ball screw 222 and are configured to move in the axial direction of the ball screw 222 by forward and reverse rotation of the ball screw 222.

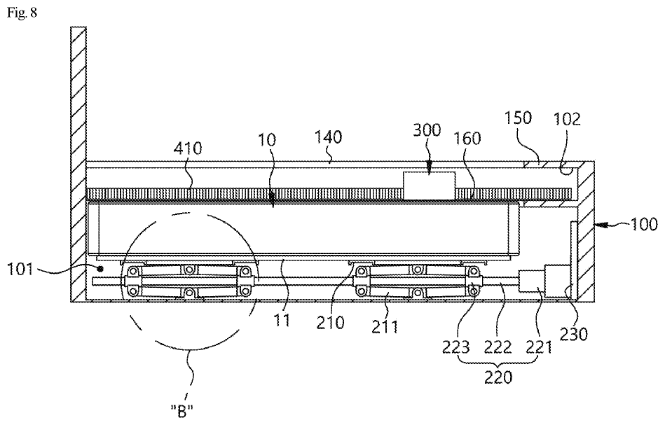

In other words, the ball housings 223 are moved along the axial direction of the ball screw 222 by the rotation of the ball screw 222 and accordingly, each link 211 is erected (refer to attached FIGS. 2 and 7) or laid down (refer to attached FIGS. 8 and 9), moving the lift frame 210 up or down. The combination of the links 211, the ball housings 223 and the ball screw(s) 222 act as a jack for lifting the lift frame 210 and thus lifting the mattress 10 disposed on the lift frame 210.

Of course, a support frame 11 may be configured to be coupled to the upper surface of the lift frame 210, and the mattress 10 may be configured to be placed on the upper surface of the support frame 11. The support frame 11 may be sized to overlap an entirety of the mattress 10 or the support frame 11 may be sized to overlap a majority of the mattress 10. Further, the lift frame 210 may be fixedly attached to the support frame 11. For example, the lift frame 210 may be removably attached to the support frame 11 via fasteners, or may be permanently attached to the support frame 11 by welding, brazing or the like. Further, the support frame 11 may be the in form of rectangular shaped frame having a plurality of openings (i.e., windows), and each lift frame 210 may have an “H” shape and may directly engage the support frame 11.

The screw rotation motor 221 may be configured to be positioned in the bottom space within the storage part 102. That is, the screw rotation motor 221 can move the mattress 10 downward as much as possible since it is not located in the accommodation part 101, also, the screw rotation motor 221 does not interfere with the movement of the cleaning module 300 located in the upper space, since it is located in the lower space in the storage part 102.

The screw rotation motor 221 may be installed to be raised and lowered in the bottom space of the storage part while being installed on a lift plate 230. Further, the lift plate 230 may be attached to the bed frame 100. That is, considering that the ball screw 222 is also moved up and down due to the rotation of the links 211 by the operation of the lift part 220, the screw rotation motor 221 may also be lifted up and down together.

Meanwhile, the lift operation part 200 may have various structures other than the structure using the above-described ball screw 222.

For example, the lift operation part 200 may have various structures using an actuator, a plurality of links, or a plurality of gears.

Next, the mattress care device according to an embodiment of the present disclosure is configured to include a cleaning module 300.

The cleaning module 300 is a device provided to clean the surface of the mattress 10.

The cleaning module 300 is positioned to be stored in the storage part 102 when not in use as shown in FIGS. 1 and 2, while being located in the accommodation part 101 outside the storage part 102 when used as shown in FIGS. 3, 4, and 8 to move back and forth.

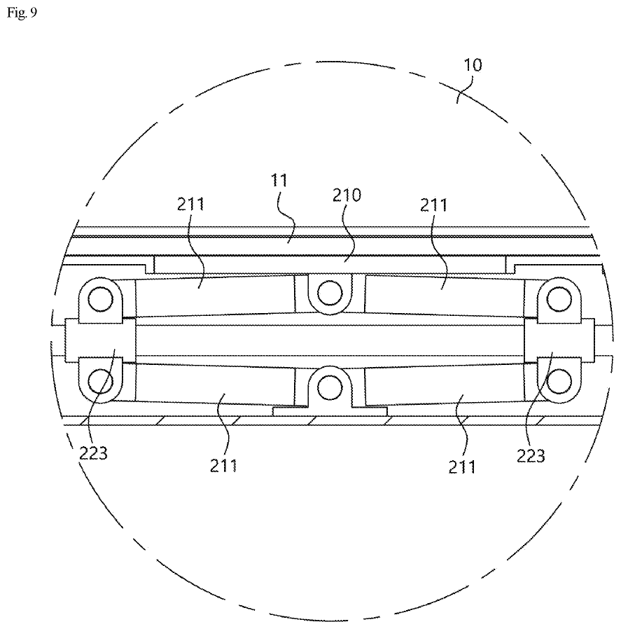

The accompanying FIGS. 10 to 16 show the structure for each direction and each state of the cleaning module 300.

As shown in these drawings, the cleaning module 300 is configured to include a case 310 forming an exterior of the cleaning module 300.

The left and right length of the case 310 is formed to be longer than the left and right width of the mattress 10, and both ends of the case are respectively connected to opposite side panels in the bed frame 100.

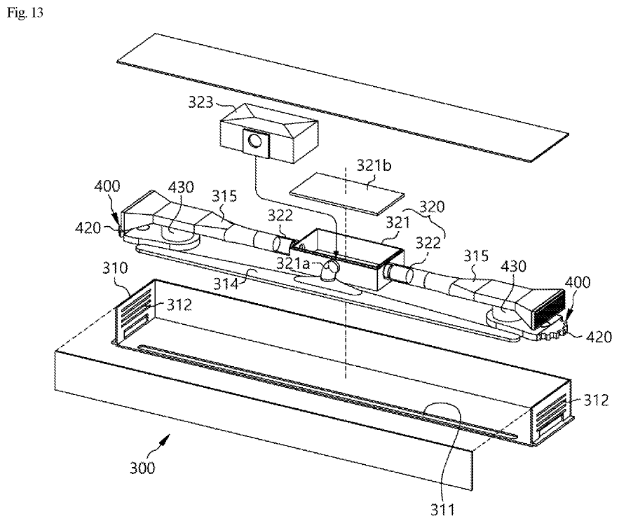

The case 310 has an inlet 311 through which air is sucked and an outlet port 312 through which air is discharged. That is, the air sucked into the case 310 through the inlet 311 can be discharged through the outlet 312, as shown in FIG. 13. The inlet 311 may extend substantially an entire width of the cleaning module 300 (e.g., of the bottom surface of the cleaning module 300).

Here, the inlet 311 may be formed along the longitudinal direction on the bottom surface of the case 310. In particular, the inlet 311 may be formed to be equal to or longer than the left and right length of the mattress 10.

The outlet 312 may be formed on at least one wall of opposite side walls of the case 310 or may be formed on at least one of the upper surfaces of both ends of the case 310.

If the outlet 312 is formed on the upper surface of the case 310, there is a fear that the user may feel uncomfortable because the exhaust air is discharged to the upper portion of the cleaning module 300. Considering this, the outlet 312 may be preferably formed on opposite side walls of the case 310 or may be preferably formed on opposite side bottom surfaces.

A filter (e.g., a HEPA filter or any type of filter) for filtering fine dust, allergens, viruses, bacteria, and like, from the discharged air may be provided at the outlet 312.

In addition, the cleaning module 300 may be configured to include a suction part 320.

The suction part 320 is a configuration for vacuum suction of foreign substances on the surface of the mattress 10, and may be configured to include a dust collector 321 and a fan motor 322 installed in (i.e., positioned in) the case 310.

The dust collector 321 is configured to collect foreign substances that are suctioned and flowed through the inlet 311 while being located on the center of the case 310.

A connection hole 321a connecting and communicating with the inside of the dust collector 321 is formed on the front wall of the dust collector 321, and the connection hole 321a is configured to be connected to the inlet 311 and a connection duct 314. Air and foreign substances sucked in through the inlet 311 are guided by the connection duct 314 and pass through the connection hole 321a.

A dust bag 323 may be removably installed inside the dust collector 321 so as to be replaceable, in this way, foreign substances in the air passing through the dust collector 321 may be filtered by the dust bag 323.

The upper surface of the dust collector 321 is configured to be openable and with an open/close cover 321b, thus enabling replacement of the dust bag 323.

The fan motor 322 is a device that generates a vacuum suction force, and may be provided on at least one side of the dust collector 321.

Preferably, as shown in FIGS. 13, 14, and 16, the fan motor 322 may be provided in two, with one fan motor 322 on each side of the dust collector 321.

The air flowing into the dust collector 321 and passing through the dust bag 323 passes through each fan motor 322, flows to both sides in the case 310, and then is discharged to through the outlet 312. Here, the air that has passed through the fan motor 322 may be discharged to the outlet 312 by being guided by the outlet duct 315.

The cleaning module 300 may include a UV-C irradiation part 330 for radiating UV-C as shown in FIG. 16. That is, the upper surface of the mattress 10 may be sterilized thanks to the UV-C irradiated by the UV-C irradiation part 330.

The UV-C irradiation part 330 may be provided on at least one portion of the bottom surface of the case 310 constituting the cleaning module 300, and preferably, it may be provided on a peripheral portion of the bottom surface of the case 310. The UV-C irradiation part 330 is shown in FIG. 16.

The UV-C irradiation part 330 may be composed of any one of a UV-C lamp and a UV-C LED, or may be composed of any type of UV light that has a sterilization effect. In an embodiment of the present disclosure, it is assumed that the UV-C irradiation part 330 is composed of a UV-C LED.

The cleaning module 300 may be provided with a proximity sensor 340 for detecting proximity to the bed frame 100. With the proximity sensor 340, the cleaning module 300 may reach a position as close as possible without colliding with the front-side panel or the rear-side panel of the bed frame 100.

The proximity sensor 340 may be formed one each on the front and rear surfaces of the cleaning module 300.

The proximity sensor 340 may be configured as a general optical sensor, or may be configured as a switch whose contact is on or off depending on the contact.

The proximity sensor 340 may be provided in the bed frame 100 rather than in the cleaning module 300.

Meanwhile, the cleaning module 300 is configured to be moved in the front-rear direction of the bed frame 100 while maintaining the same height by receiving the support of a guide part 160.

The guide part 160 may be formed on at least one of the opposite surfaces between the bed frame 100 and the cleaning module 300.

In an embodiment of the present disclosure, it is suggested that both ends of the case 310 constituting the cleaning module 300 may be raised while the guide parts 160 protrude inward from the inner surfaces of opposite sides of the bed frame 100. That is, the cleaning module 300 may be moved back and forth with both ends mounted on each of the guide parts 160, respectively.

Next, the mattress care device according to an embodiment of the present disclosure is configured to include a cleaner moving part 400.

The cleaner moving part 400 is configured such that the cleaning module 300 is moved in the front-rear direction within the bed frame 100.

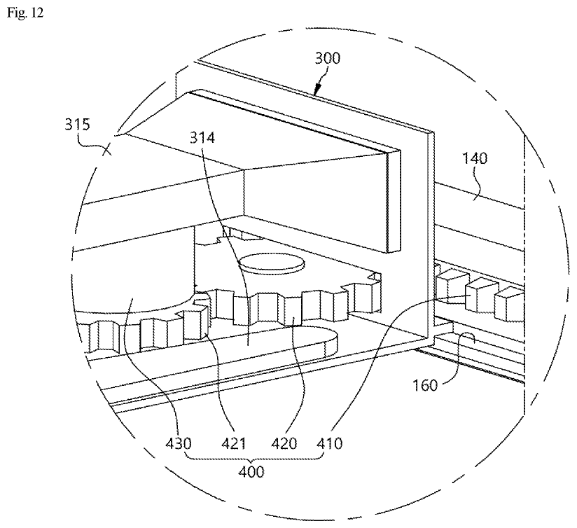

The cleaner moving part 400 may have a rack and pinion structure as shown in FIG. 12.

For example, the rack gears 410 are formed along opposite side panels 120 in the bed frame 100 and pinion gears 420 engaged with the respective rack gears 410 are provided on both sides (i.e., opposing sides) of the cleaning module 300, thus the cleaning module 300 may be moved in the front-rear direction within the bed frame 100.

Here, the pinion gears 420 are installed to penetrate opposite side walls of the case 310 so that parts thereof is exposed to the outside of the case 310, and gear driving motors 430 (i.e., two opposing gear driving motors 430 provided at opposite sides of the case 310) for driving the pinion gears 420 are provided on both sides of the case 310.

Each gear driving motor 430 may be installed directly connected to the pinion gear 420, or as shown in the embodiment, it may be installed to rotate the pinion gear 420 using another power transmission gear 421.

Next, the mattress care device according to an embodiment of the present disclosure is configured to include a controller 500.

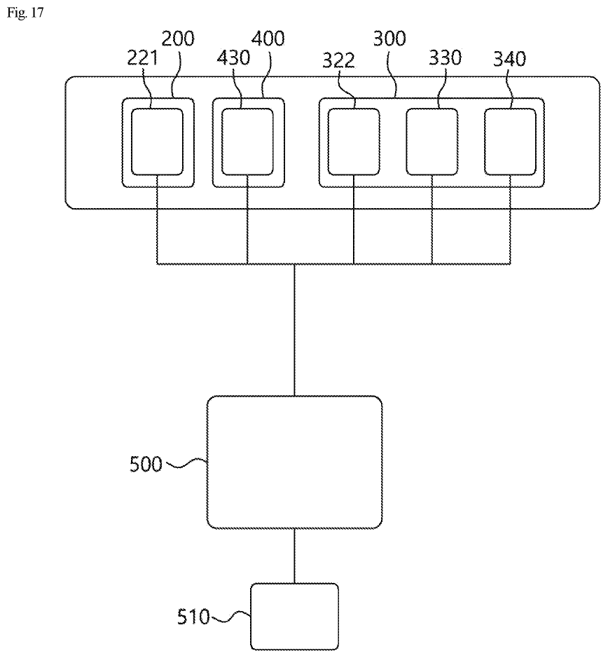

The controller 500, as shown in FIG. 17, may be configured to control the operation of the lift operation part 200, the cleaning module 300, and the cleaner moving part 400.

That is, the controller 500 may control the lifting operation of the mattress 10 while controlling the screw rotation motor 221, may control the forward and backward movement of the cleaning module 300 by controlling the operation of the gear driving motor(s) 430, and may control the operation of the cleaning module 300 by controlling the fan motor 322 and the UV-C irradiation part 330.

The controller 500 may be configured to include an operation part 510 for manual operation. The operation part 510 may be provided in the bed frame 100 or in the cleaning module 300.

Preferably, the operation part 510 may be provided on any one of the outer surfaces of the bed frame 100, and the controller 500 may be provided in the lift operation part 200 or in the cleaning module 300.

The controller 500 may be configured to be operated through a user's mobile terminal, home network, or other online control (e.g., cellular network, Bluetooth™ or the like).

Especially, the controller 500 may be configured to control the moving speed of the cleaning module 300 differently according to the operation of the UV-C irradiation part 330.

That is, when the irradiation part 330 is emitted so that UV-C is irradiated, the moving speed of the cleaning module 300 or a moving frame 200 is controlled to move slower than the moving speed when the UV-C irradiation part 330 does not emit light. Accordingly, UV-C may be sufficiently irradiated to the mattress 10 to obtain a sterilization effect, and when only suction cleaning is performed without sterilization cleaning, the cleaning time may be shortened.

In addition, the controller 500 may be configured to control the front and rear movement of the cleaning module 300 to be stopped when a sensing signal is received from the proximity sensor 340 provided in the cleaning module 300.

In the following, the cleaning process of the mattress 10 using the mattress care device of the present disclosure described above will be described in more detail.

To begin with, in the case in which the cleaning of the mattress 10 is not performed, the mattress 10 is maintained in a state in which at least a portion protrudes from the accommodation part 101 of the bed frame 100 by the lift operation part 200.

The cleaning module 300 is stored in a state located in the storage part 102 of the bed frame 100, and the storage part 102 is prevented from being exposed to the outside by the shielding plate 150, the shielding plate 150 forming the upper surface of the bed frame 100, so that the cleaning module 300 is not visible to the outside. This is as shown in the attached FIGS. 1-4.

In this initial state, when a preset cleaning cycle arrives (i.e. is automatically performed) or a user's operation through the operation part 510 occurs and a cleaning request is made, the controller 500 controls the lift operation part 200 to control the mattress 10 to move down.

That is, the ball screw 222 is rotated while the screw rotation motor 221 is driven under the control of the controller 500, thereby the lift frame 210 is lowered and the mattress 10 placed on the upper surface of the lift frame 210 is also lowered (i.e., the mattress 10 is lowered with the lowering of the lift frame 210) and the mattress 10 is accommodated in the accommodation part 101 while descending and once fully descended. This is as shown in attached FIGS. 7 and 8.

The lowering of the mattress 10 is continued until the upper surface of the mattress 10 is positioned at the same height as the bottom surface of the cleaning module 300 or is positioned lower than the bottom surface of the cleaning module 300. The descending distance of the mattress 10 may be preset by the user or controlled to be moved automatically using a sensor.

When the lowering of the mattress 10 is completed, the cleaning module 300 is moved forward (i.e., toward the front side of the bed frame) while the operation control of the cleaner moving part 400 is performed.

That is, the gear driving motors 430 constituting the cleaner moving part 400 are driven to rotate the respective pinion gear 420, as a result, the pinion gears 420 are moved in the direction in which the rack gears 410 while being engaged with the rack gear 410. This is as shown in the attached FIGS. 3, 4 and 12.

At this time, the cleaning module 300 is moved with its both ends mounted on the guide part 160 as shown in FIG. 12.

In addition, when the cleaning module 300 is moved forward, the fan motor 322 of the suction part 320 may be controlled to operate together, by the controller 500.

The fan motor 322 is controlled, by the controller 500, to operate from when the cleaning module 300 is moved, or from when the cleaning module 300 leaves the storage part 102 and is positioned on the upper surface of the rear side of the mattress 10.

When the fan motor 322 is operated, an air suction force is generated through the inlet 311.

By the air suction force generated in this way, foreign substances existing on the surface of the mattress 10 are introduced into the dust bag 323 in the dust collector 321 after sequentially passing through the inlet 311, the connecting duct 314, and the connection hole 321a.

Thereafter, the foreign substances are filtered in the dust bag 323 and the air flows out of the dust bag 323 to both sides (i.e., opposing sides) of the case 310 and then is discharged to the outside of the cleaning module 300 through the outlet 312. Since a filter (e.g., a HEPA filter) is provided at the outlet 312, fine dust contained in the air passing through the outlet 312 is filtered by the filter.

In this way, the cleaning module 300 is moved from the rear end to the front end of the mattress 10 to suck the foreign substances present on the upper surface of the mattress 10 and collect the dust in the dust bag 323 disposed in the dust collector 321.

The controller 500 confirms that the cleaning module 300 reaches the front end of the mattress 10 through the proximity sensor 340, and when the cleaning module 300 reaches the front end of the mattress 10, the operation of the gear driving motor 430 constituting the cleaner moving part 400 is controlled, by the controller 500, to stop.

Thereafter, the controller 500 cleans the surface of the mattress 10 again by controlling the cleaning module 300 to move in the reverse direction (moving toward the rear side of the mattress 10). Of course, when the forward movement of the cleaning module 300 is terminated and the cleaning module 300 is returned to an initial position (i.e., a position inside of the storage part 102), the suction part 320 is not operated and may be controlled, by the controller 500, to return to its initial position quickly.

Meanwhile, during the cleaning of the mattress 10 described above, the UV-C irradiation part 330 of the cleaning module 300 may be operated according to the user's setting or operation.

Of course, the UV-C irradiation part 330 may be controlled, by the controller 500, to operate independently, not simultaneously with the suction part 320, or may be controlled to operate only when the cleaning module 300 moves forward or backward, or can be controlled to simultaneously operate when the suction part 320 is operated.

In this way, when the UV-C irradiation part 330 is operated, UV-C is radiated to the surface of the mattress 10, thereby killing pests (i.e., viruses) and/or bacteria present in the mattress 10.

In particular, when the UV-C irradiation part 330 of the cleaning module 300 is operated, the cleaning module 300 is controlled to move more slowly than when only the suction part 320 is operated. That is, when the UV-C irradiation part 330 is operated, the cleaning module 300 is controlled to move more slowly so that the sterilization effect using UV-C may be sufficiently obtained.

Meanwhile, when the above-described cleaning of the surface of the mattress 10 is completed, the cleaning module 300 is returned into the storage part 102, and then the mattress 10 is moved upward from the accommodation part 101 by the operation of the lift operation part 200 to protrude from the accommodation part 101.

Therefore, the storage part 102 and the accommodation part 101 are covered by the mattress 10 so that the cleaning module 300 stored in the storage part 102 may also be stored in a state in which its exposure to the outside is prevented.

If the dust bag 323 is to be replaced, the open/close cover 321b is opened and the upper surface of the dust collector 321 is opened.

To sum up, the mattress care device of the present disclosure can automatically sterilize and vacuum the mattress 10, thereby enabling continuous care of the mattress 10.

Also, in the case of the mattress care device of the present disclosure, the user does not feel discomfort because the cleaning module 300 does not exposed to the outside while being stored in the storage part 102 when the cleaning module 300 does not perform cleaning of the mattress 10, and no interference occurs when the user gets on and off the bed.

In addition, in the case of the mattress care device of the present disclosure, since the device cleans the mattress 10 while the sterilizer 300 operates in a state located in the bed frame 100, it is possible to freely design the exterior of the bed frame 100 or the exterior of the mattress 10.

Also, in the case of the mattress care device of the present disclosure, the operation part of the cleaning module 300 is not exposed to the outside, so that the malfunction caused by the entrapment of foreign substances can be prevented.

In addition, in the case of the mattress care device of the present disclosure, the height adjustment of the lift frame 210 is possible as the lift part 220 constituting the lift operation part 200 is configured using a ball screw 222 that is rotated by the driving of the screw rotation motor 221, thus making the mattress care device suitable for use on any mattress regardless of the thickness of the mattress 10.

Moreover, the mattress care device of the present disclosure is not limited to being implemented only with the structure of the above-described embodiment.

For example, the mattress care device of the present disclosure may include a vibration part 250 in the cleaning module 300. That is, as the vibration part 250 may be additionally provided, the surface of the mattress 10 through which the cleaning module 300 passes is vibrated by the vibration part 250, and foreign substances such as dust adhering to the surface of the mattress 10 are dropped so that they can be easily sucked.

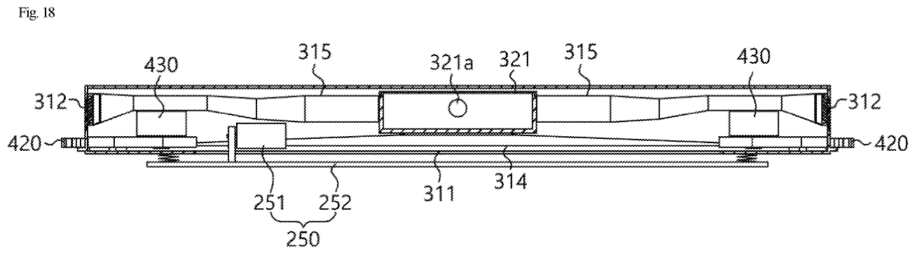

The vibration part 250 includes a vibration source 251, such as an ultrasonic vibrator or a vibration motor, and a vibration plate 252 vibrating up and down by the operation of the vibration source 251 as shown in the attached FIG. 18.

Various embodiments described herein may be implemented in a computer-readable medium using, for example, software, hardware, or some combination thereof. For example, the embodiments described herein may be implemented within one or more of Application Specific Integrated Circuits (ASICs), Digital Signal Processors (DSPs), Digital Signal Processing Devices (DSPDs), Programmable Logic Devices (PLDs), Field Programmable Gate Arrays (FPGAs), processors, controllers, micro-controllers, microprocessors, other electronic units designed to perform the functions described herein, or a selective combination thereof. In some cases, such embodiments are implemented by the controller. That is, the controller is a hardware-embedded processor executing the appropriate algorithms (e.g., flowcharts) for performing the described functions and thus has sufficient structure. Also, the embodiments such as procedures and functions may be implemented together with separate software modules each of which performs at least one of functions and operations. The software codes can be implemented with a software application written in any suitable programming language. Also, the software codes can be stored in the memory and executed by the controller, thus making the controller a type of special purpose controller specifically configured to carry out the described functions and algorithms. Thus, the components shown in the drawings have sufficient structure to implement the appropriate algorithms for performing the described functions.

As such, the mattress care device of the present disclosure may be implemented in various forms.

The present invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A mattress care device, comprising:
a bed frame including:
an accommodation part configured to accommodate a mattress; and
a storage part communicating with the accommodation part;
a lift operation part configured to move the mattress along a vertical direction with respect to the bed frame;
a cleaning module configured to:
move to be stored in the storage part when not in use; and
move in a longitudinal direction of the mattress in the accommodation part when in use;
a cleaner moving part configured to move the cleaning module along the longitudinal direction of the mattress; and
a controller configured to control operations of the lift operation part, the cleaning module, and the cleaner moving part.

2. The mattress care device of claim 1, wherein the bed frame is in the form of a box and includes:
an open top; and
opposing side panels.

3. The mattress care device of claim 1, wherein a width of the cleaning module is configured to be larger than a width of the mattress, and
wherein opposing ends of the cleaning module are respectively connected to opposing side panels of the bed frame.

4. The mattress care device of claim 2, wherein the bed frame further includes an edge part extending from the first and second side panels towards a center of the bed frame and protruding inward around a periphery of the open top of the bed frame.

5. The mattress care device of claim 2, wherein the bed frame further includes a shielding plate covering an upper surface of the storage part.

6. The mattress care device of claim 1, wherein the storage part is formed up to a rear panel of the bed frame and communicates with a foot-side portion of the accommodation part.

7. The mattress care device of claim 6, wherein the storage part is configured to completely accommodate the cleaning module when the cleaning module is not in use.

8. The mattress care device of claim 1, wherein the lift operation part includes:
a lift frame configured to accommodate the mattress; and
a lift part configured to raise and lower the lift frame along the vertical direction.

9. The mattress care device of claim 8, wherein the lift part includes:
at least one ball screw;
at least one link connected to the lift frame; and
a screw rotation motor configured to rotate the at least one ball screw to move the at least one link along the vertical direction.

10. The mattress care device of claim 1, wherein the lift operation part is configured to position an upper surface of the mattress at substantially a same height, in the vertical direction, as a bottom surface of the cleaning module while being lower than an upper surface of the bed frame, in response to the cleaning module being in use.

11. The mattress care device of claim 10, wherein the lift operation part is configured to position an upper surface of the mattress higher than an upper surface of the bed frame, in response to the cleaning module not being in use.

12. The mattress care device of claim 1, wherein the cleaning module includes:
an inlet formed on a bottom surface of the cleaning module;
a fan motor configured to generate a vacuum suction force; and
a dust collector configured to collect foreign substances sucked through the inlet.

13. The mattress care device of claim 12, wherein at least one wall of the cleaning module is provided with an outlet port through which air is discharged by the fan motor.

14. The mattress care device of claim 12, wherein the cleaning module includes an ultraviolet-c (UV-C) irradiation part on the bottom surface of the cleaning module, and
wherein the UV-C irradiation part is configured to radiate UV-C light onto a top surface of the mattress.

15. The mattress care device of claim 1, wherein the cleaner moving part includes:
a first rack gear extending along a first side panel among two opposing side panels of the bed frame;
a second rack gear extending along a second side panel among the two opposing side panels of the bed frame;
a first pinion gear provided on a first side of the cleaning module among two opposite sides of the cleaning module, the first pinion gear engaging the first rack gear;
a second pinion gear provided on a second side of the cleaning module among the two opposite sides of the cleaning module, the second pinion gear engaging the second rack gear;
a first gear driving motor provided on the first side of the cleaning module, the first gear driving motor being configured to drive the first pinion gear to cause the first pinion gear to rotate with respect to the first rack gear; and
a second gear driving motor provided on the second side of the cleaning module, the second gear driving motor being configured to drive the second pinion gear to cause the second pinion gear to rotate with respect to the second rack gear.

16. A mattress care device, comprising:
a bed frame having an accommodation part configured to accommodate a mattress;
a lift operation part configured to move the mattress in a vertical direction with respect to the bed frame;
a cleaning module configured to clean a surface of the mattress while moving in a longitudinal direction of the mattress;
a cleaner moving part configured to move the cleaning module in the longitudinal direction of the mattress; and
a controller configured to control operations of the lift operation part, the cleaning module, and the cleaner moving part.

17. The mattress care device of claim 16, wherein the cleaning module includes:
an inlet formed on a bottom surface of the cleaning module;
at least one fan motor configured to generate a vacuum suction force; and
a dust collector configured to collect foreign substances sucked through the inlet.

18. The mattress care device of claim 17, wherein the inlet extends substantially an entire width of the cleaning module.

19. The mattress care device of claim 17, wherein the cleaning module further includes opposing outlets extending from opposing sides of the dust collector,
wherein the dust collector is located at a center of the cleaning module, and
wherein the at least one fan motor includes two fan motors.

20. The mattress care device of claim 19, wherein the two fan motors are provided at the opposing sides of the dust collector,
wherein the two fan motors are configured to generate an air flow through the inlet, then through the dust collector, and then out through the opposing outlets, and
wherein the dust collector is connected to the inlet by a connection duct.
